(19) United States
(12) Patent Application Publication
(10) Pub. No.: US 2014/0261545 A1
(43) Pub. Date: Sep. 18, 2014

(54) APPARATUS FOR WIPING ANGLED WINDOW OF ENDOSCOPE

(71) Applicant: ACCLARENT, INC., Menlo Park, CA (US)

(72) Inventors: Thomas R. Jenkins, Alameda, CA (US); Eric Goldfarb, Belmont, CA (US)

(21) Appl. No.: 13/804,684

(22) Filed: Mar. 14, 2013

(51) Int. Cl. A61B 1/12

(52) U.S. Cl. CPC A61B 1/122
USPC 134/8; 15/104.05; 15/3.5

(57) ABSTRACT

An endoscope cleaning system comprises a sheath and a window cleaning feature. The sheath is configured to receive a shaft of an endoscope. The window cleaning feature is configured to clean the window of the endoscope. The window cleaning feature may include an absorptive material that is configured to soakingly wipe liquids and/or debris from the window when the absorptive material is passed over the window. The window cleaning feature may also include a hydrophilic wiper that is configured to drivingly wipe liquids and/or debris away from the window when the wiper is passed over the window. The lends cleaning feature may also include a fluid washing feature, a suction feature, and/or a pressurized gas feature to assist in cleaning the window of the endoscope.

### BACKGROUND

In some instances, it may be desirable to dilate an anatomical passageway in a patient. This may include dilation of ostia of paranasal sinuses (e.g., to treat sinusitis), dilation of the larynx, dilation of the Eustachian tube, dilation of other passageways within the ear, nose, or throat, etc. One method of dilating anatomical passageways includes using a guide wire and catheter to position an inflatable balloon within the anatomical passageway, then inflating the balloon with a fluid (e.g., saline) to dilate the anatomical passageway. For instance, the expandable balloon may be positioned within an ostium at a paranasal sinus and then be inflated, to thereby dilate the ostium by remodeling the bone adjacent to the ostium, without requiring incision of the mucosa or removal of any bone. The dilated ostium may then allow for improved drainage from and ventilation of the affected paranasal sinus. A system that may be used to perform such procedures may be provided in accordance with the teachings of U.S. Pub. No. 2011/0004057, entitled “Systems and Methods for Transnasal Dilation of Passageways in the Ear, Nose or Throat,” published Jan. 6, 2011, the disclosure of which is incorporated by reference herein. An example of such a system is the Relieva® Spin Balloon Sinuplasty™ System by Acclarent, Inc. of Menlo Park, Calif.

A variable direction view endoscope may be used with such a system to provide visualization within the anatomical passageway (e.g., the ear, nose, throat, paranasal sinuses, etc.) to position the balloon at desired locations. A variable direction view endoscope may enable viewing along a variety of transverse viewing angles without having to flex the shaft of the endoscope within the anatomical passageway. Such an endoscope that may be provided in accordance with the teachings of U.S. Pub. No. 2010/0030031, entitled “Swing Prism Endoscope,” published Feb. 4, 2010, the disclosure of which is incorporated by reference herein. An example of such an endoscope is the Acclarent Cyclops™ Multi-Angle Endoscope by Acclarent, Inc. of Menlo Park, Calif.

In some instances, it may be desirable to provide a cleaning system for use with the endoscope to maintain visualization within an anatomical passageway. While several systems and methods have been made and used to clean an endoscope for use in an anatomical passageway, it is believed that no one prior to the inventors has made or used the invention described in the appended claims.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements and in which:

FIG. 1 depicts a side view of an exemplary dilation catheter system;

FIG. 2 depicts a perspective view of an endoscope for use with the dilation catheter system of FIG. 1;

FIG. 3 depicts a cross sectional view of a distal end of the endoscope of FIG. 2, showing viewing ranges of the endoscope;

FIG. 4 depicts a perspective view of an exemplary cleaning system;

FIG. 5A depicts a cross-sectional side view of a portion of the cleaning system of FIG. 4, with a button in a non-actuated state;

FIG. 5B depicts a cross-sectional side view of a portion of the cleaning system of FIG. 4, with the button actuated;

FIG. 6 depicts a cross-sectional side view of an exemplary alternative distal end configuration for the cleaning system of FIG. 4, in a partially actuated position;

FIG. 7 depicts an enlarged perspective view of another exemplary alternative distal end configuration for the cleaning system of FIG. 4, in a partially actuated position;

FIG. 8A depicts a side view of an exemplary alternative version of a wiper made of a shape forming material;

FIG. 8B depicts a side view of the wiper of FIG. 8A with an endoscope extended through a sheath of the wiper;

FIG. 9 depicts a perspective view of an exemplary distal end of a wash and suction device; and

FIG. 10 depicts a cross sectional end view of the wash and suction device of FIG. 9.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It will be appreciated that the terms “proximal” and “distal” are used herein with reference to a clinician gripping a handpiece assembly. Thus, an end effector is distal with respect to the more proximal handpiece assembly. It will be further appreciated that, for convenience and clarity, spatial terms such as “top” and “bottom” also are used herein with respect to the clinician gripping the handpiece assembly. However, surgical instruments are used in many orientations and positions, and these terms are not intended to be limiting and absolute.

It is further understood that any one or more of the teachings, expressions, versions, examples, etc. described herein may be combined with any one or more of the other teachings, expressions, versions, examples, etc. that are described herein. The following-described teachings, expressions, versions, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

I. Overview of Exemplary Dilation Catheter System

FIG. 1 shows an exemplary dilation catheter system (1), which may be used to dilate the ostium of a paranasal sinus; or to dilate some other anatomical passageway (e.g., within the ear, nose, or throat, etc.). Dilation catheter system (1) of this example comprises a dilation catheter (10), a dilator (14), a guide catheter (70), and an inflator (50). System (1) may be configured in accordance with at least some of the teachings of U.S. Patent Pub. No. 2011/0004057, entitled “Systems and Methods for Transnasal Dilation of Passageways in the Ear, Nose or Throat,” published Jan. 6, 2011, the disclosure of which is incorporated by reference herein. In some versions, at least part of system (1) is configured similar to the Relieva® Spin Balloon Sinuplasty™ System by Acclarent, Inc. of Menlo Park, Calif. Dilator (14) of the present example is coupled to a distal end of dilation catheter (10), and inflator (50) is coupled to a proximal portion of dilation catheter (10), such that dilation catheter (10) provides fluid communication between inflator (50) and dilator (14). Dilation catheter (10) is slidably positioned through guide catheter (70).

In a dilation procedure, guide catheter (70) may first be positioned near the targeted anatomical passageway. Guide catheter (70) is initially inserted into the nose of the patient and is advanced to a position that is within or near the ostium to be dilated. After guide catheter (70) has been positioned, the operator may insert the distal end of a guidewire (GW) into the proximal end of guide catheter (70) and may advance the guidewire (GW) through guide catheter (70) such that a distal portion of the guidewire (GW) passes through the sinus ostium (SO) and becomes coiled within the sinus cavity. Thereafter, the proximal end of the guidewire (GW) is inserted into the distal end of dilation catheter (10), and dilation catheter (10) (with dilator (14) in a non-expanded state) is advanced over the guidewire (GW) and through guide catheter (70) to a position where dilator (14) is positioned within the sinus ostium (SO), or other targeted anatomical passageway.

After dilator (14) has been positioned within the ostium, dilator (14) may be inflated, thereby dilating the ostium. To inflate dilator (14), plunger (54) may be actuated to push saline from inflator (50) through dilation catheter (10) into dilator (14). The transfer of fluid expands dilator (14) to an expanded state to open or dilate the targeted anatomical passageway. Dilator (14) may be inflated to a volume size to achieve about 10 to about 12 atmospheres. Dilator (14) may be held at this volume for a few seconds to sufficiently open the ostium or targeted anatomical passageway. Dilator (14) may then be deflated (or returned to a non-expanded state) by actuating plunger (54) of inflator (50) to bring the saline back to inflator (50). Dilator (14) may be repeatedly inflated and deflated in different ostia and/or other targeted anatomical passageways. Thereafter, dilation catheter (10), guidewire (GW), and guide catheter (70) may be removed from the patient.

II. Overview of Exemplary Endoscope

In some uses of system (1) shown in FIG. 1, an endoscope may be placed in the ear, nose, or throat to view all or part of the procedure. For instance, the successful positioning of dilator (14) and dilation of the ostium may be confirmed visually using an endoscope. FIG. 2 shows an exemplary endoscope (110) for use with the dilation catheter system of FIG. 1 or in any other suitable type of context. In some versions, endoscope (110) may be configured in accordance with at least some of the teachings of U.S. Pub. No. 2010/0030031, entitled “Swing Prism Endoscope,” published Feb. 4, 2010, the disclosure of which is incorporated by reference herein. Endoscope (110) of the present example comprises an elongate shaft (130) with a distal end (170) and a proximal end (171). Proximal end (171) of shaft (130) is attached to handle (152). Distal end (170) of endoscope (110) may be placed within the desired anatomical passageway to provide visualization to the user.

A. Exemplary Shaft

As shown in FIG. 3, shaft (130) houses an image fiber bundle or optic fibers (154) that extend coaxially through the center of shaft (130). Shaft (130) may also include light transmitting fibers disposed about the periphery of optic fibers (154). In some versions, shaft (130) is a braided polyimide sheathing that has a maximum outer diameter of about 0.0375 inches and a length of about two feet. Endoscope shaft (130) may have an outer diameter of approximately 4 mm and a working length of about 175 mm. Shaft (130) has rounded surfaces thus making the assembly atraumatic in use. Optic fibers (154) are made up of 10,000 thin image fibers, and the light transmitting fibers are illumination fibers with a diameter of between about 0.008 and 0.020 inches, with a minimum lux of about 10,000. In other versions, endoscope (110) uses rod lens technology instead of image fiber bundles. Of course, any of the above-noted parameters may be varied as desired.

As shown in FIG. 3, distal end (170) of shaft (130) comprises a swing prism (172), a lens (174), a window (175), an actuator (178), and a resilient member (176). Swing prism (172) is mounted for rotation between resilient member (176) and actuator (178). In particular, swing prism (172) is pivotable about an axis (171), which is transverse to the longitudinal axis of shaft (130). Actuator (178) may comprise any suitable structure, such as a push-pull cable, a rod, a beam, etc., that is operable to communicate longitudinal motion along shaft (130). Actuator (178) extends from distal portion (170) of endoscope (110) to a proximal portion. Actuator (178) is driven by rotation of proximal dial (108) relative to handle (152). By way of example only, a nut and lead screw assembly (not shown) may be used to convert rotary motion of proximal dial (108) into longitudinal motion of actuator (178). Suitable actuator (178) configurations will be apparent to one with ordinary skill in the art in view of the teachings herein. Actuator (178) may be used to rotate swing prism (172) within distal end (170). Swing prism (172) is positioned adjacent to a window (175), which may be formed by glass, crystal, plastic, and/or any other suitable material. A self-focusing lens (174) is positioned between swing prism (172) and fiber bundle (154). Images may be captured and received through window (175) and transmitted through swing prism (172) and self-focusing lens (174) to image fiber bundle (154). In the present example, swing prism (172) provides a seventy degree field of view throughout a viewing range of zero degrees to ninety five degrees by manipulating actuator (178). Alternatively, any other suitable viewing range may be provided.

Distal end (170) of endoscope shaft (130) is shown with angular measurements in FIG. 3. In describing FIG. 3, “field of view” means the angular width/height viewed at any one time via endoscope (110), “direction of view” means the direction in which the center of view or line of sight is pointing at any one time (also can be called the “degree of view” as in “variable degree of view endoscope”) and “total range of view” means the total angular distance across which endoscope (110) can view when the swing prism (172) is moved from one extreme direction of view to the opposite extreme direction of view. The angles referred to are in relation to the longitudinal axis of endoscope shaft (130), which is the zero angle.

In the present example, endoscope (110) has a range of directions of view from about −5° to about 150°, such as from about 0° to about 120°, or from about 5° to about 100°, or from about 10° to about 90°. In some versions, endoscope (110) has a field of view from about 50° to about 100°, such as from about 60° to about 70°. From the ranges of the directions of view and the fields of view, the total ranges of view may be determined. For example, if endoscope (110) has directions of view ranging from about 5° to about 100° and a field of view of about 60°, the total range of view would be from about −25° to about 130°. If the ranges of directions of view were instead from about 0° to about 120° and the field of view were about 60°, then the total range of view would be from about −30° to about 150°. Endoscope (110) may have any of a number of different combinations and ranges of directions of view, fields of view and total ranges of view.

The images collected by image fiber bundle (154) may be transmitted to a monitor (described below) to thereby provide the operator with visualization of the procedure being performed. In some versions, endoscope (110) is compatible with a 300 Watt Xenon source and is configured with a universal light guide connector, thus making the assembly useable with conventionally available devices.

B. Exemplary Handle

As shown in FIG. 2, proximal end (171) of shaft (130) is coupled to a handle (152). Handle (152) comprises dials (108, 112), and light post (109). A proximal dial (108) is disposed on handle (152) of endoscope (110) for controlling the rotation of swing prism (172). Proximal dial (108) has a circular configuration and includes ridges that provide leverage for turning proximal dial (108) to a desired position. Handle (152) includes indicia (107) adjacent the proximal dial (108) to provide information relative to the direction of view of swing prism (172). As shown, indicia (107) adjacent to proximal dial (108) indicates the direction of view of swing prism (172) anywhere from 0 degrees to 180 degrees. A distal dial (112) is disposed on handle (152) to control rotation of endoscope shaft (130) relative to handle (152), about the longitudinal axis of shaft (130). A marker (114) is shown on distal dial (112) to indicate the relative position of endoscope shaft (130) and window (175). Rotating distal dial (112) allows endoscope (110) to view its surroundings in a full three-hundred and sixty degrees of rotation. Having a rotating distal dial (112) that rotates endoscope shaft (130) without rotating the entire handle (152) may allow for rotation of endoscope shaft (130) without rotating post (109). Post (109) extends from handle (152) to connect wiring for optic fibers (154).

III. Overview of Exemplary Wiping System

During procedures, endoscope (110) may lose visual clarity because of debris, blood, and/or mucus adhering to distal end (170). Surgeons or users may tend to remove endoscope (110) from the patient frequently to clean the distal end (170). Accordingly, it may be desirable to provide a wiping system to clear debris from distal end (170) of endoscope (110) without having to remove endoscope (110) from the patient. An actuation assembly may be provided with the wiping system to selectively clear debris from distal end (170) of endoscope (110). The examples below include merely illustrative versions of a wiping system that may be readily incorporated with endoscope (110). The below wiping features and techniques may also be combined with the washing features and techniques of U.S. patent application Ser. No. [ATTORNEY DOCKET NO. ACC5053USPSP.0600265], entitled “Apparatus for Flushing Angled Window of Endoscope,” filed on even date herewith, the disclosure of which is incorporated by reference herein.

A. Exemplary Wiping Sheath

FIG. 4 shows an exemplary cleaning system (160) for use with an endoscope (110), such as the one shown in FIG. 2. In particular, shaft (130) of endoscope (110) may be inserted through sheath (188) of cleaning system (160), such that window (175) is positioned distal to an absorptive material (190) at the distal end of sheath (188). As will be described in greater detail below, cleaning system (160) is operable to selectively advance absorptive material (190) relative to shaft (130) of endoscope (110), to thereby wipe blood, mucus, debris, etc., away from window (175) while window (175) is positioned within a patient. This wiping may be repeated several times during a medical procedure, without having to remove endoscope (110) from the patient and without having to otherwise reposition endoscope (110) within the patient.

Cleaning system (160) of the present example further includes a camming button (180) and a pair of opposing frustoconical members (182, 184). Button (180) and frustoconical members (182, 184) are positioned coaxially about shaft (130). Sheath (188) extends distally from distal frustoconical member (184) and fits shaft (130) snugly while still being capable of translating relative to shaft (130). In some versions, sheath (188) is formed of a flexible material. Sheath (188) distally terminates at absorptive material (190). Proximal frustoconical member (182) couples with dial (112) and/or proximal end (171) of shaft (130), such that proximal frustoconical member (182) does not translate relative to shaft (130) during operation of cleaning system (160). By way of example only, proximal frustoconical member (182) may couple with dial (112) through a bayonet fitting.

Button (180) is operable to be pressed by the user to advance sheath (188) distally along shaft (130). As shown in FIGS. 5A-5B, button (180) may be pressed downwardly to advance sheath (188) distally, though it should be understood that button (180) of the present example may also be pressed in any direction transverse to the longitudinal axis of shaft (130) in order to advance sheath (188) distally. Button (180) is in the form of a ring having outwardly presented camming surfaces (181) that engage complementary surfaces (183, 185) of frustoconical members (182, 184), such that button (180) increases separation between frustoconical members (182, 184) when button (180) is moved transversely relative to shaft (130) and frustoconical members (182, 184). FIG. 5A shows button (180) in a non-actuated state. In this configuration, absorptive material (190) is positioned proximal to window (175). FIG. 5B shows button (180) in an actuated state. In this configuration, interaction between the proximal camming surface (181) of button (180) and camming surface (183) of proximal frustoconical member (182) drives button (180) distally. In addition, interaction between the distal camming surface (181) of button (180) and camming surface (185) of distal frustoconical member (184) drives distal frustoconical member (184) distally. This in turn drives sheath (188) and absorptive material (190) distally, such that absorptive material (190) passes over window (175) to clear blood, mucus, debris, etc. from window (175).

As also shown in FIGS. 5A-5B, a coil spring (186) joins frustoconical members (182, 184) together. Coil spring (186) is configured to bias frustoconical members (182, 184) toward each other. When button (180) is actuated to advance distal frustoconical member (182) relative to proximal frustoconical member (184), coil spring (186) extends under stress. When the operator subsequently releases button (180), the resilient bias of coil spring (186) pulls distal frustoconical member (184) proximally back to the position shown in FIG. 5A, which in turn retracts sheath (188) and apsorptive material (190) proximally to reveal a cleaned window (175). It should also be understood that interaction between camming surfaces (181, 183, 185) also drives button (180) back to the non-actuated position shown in FIG. 5A when coil spring (186) pulls distal frustoconical member (184) back to a proximal position. Other suitable variations of coil spring (186) will be apparent to those of ordinary skill in the art in view of the teachings herein; as will be other ways in which distal frustoconical member (184) may be returned to a proximal position. By way of example only, distal frustoconical member (184) may be returned to a proximal position manually, such as by the operator directly pulling distal frustoconical member (184) back to the proximal position. In any case, it should be understood that absorptive material (190) may provide additional wiping of window (175) as sheath (188) is retracted from the distal position back to the proximal position.

In the present example, absorptive material (190) is elastic and hydrophilic. Absorptive material (190) may be formed of cloth and/or any other suitable material as will be apparent to those of ordinary skill in the art in view of the teachings herein. Absorptive material (190) may absorb and retain blood, mucus, debris, etc., while cleaning system (160) remains coupled with endoscope (110). Absorptive material (190) may use capillary-type action and/or attractive electromagnetic forces to draw fluid or other debris into absorptive material (190). Absorptive material (190) may have a contour that complements the contour of window (175). In addition or in the alternative, absorptive material (190) may have elastic properties that enable it to conform to the contour of window (175) as absorptive material (190) is advanced over window (175). By way of example only, absorptive material (190) may include fibers formed of nitinol or thermoplastic material. In addition or in the alternative, absorptive material (190) and/or sheath (188) may further comprise a mesh material or flexible rods integrated with absorptive material (190) for reinforcement, to prevent absorptive material (190) and/or sheath (188) from bucking or collapsing during longitudinal translation. It should be understood that sheath (188) may have the same or substantially the same properties as absorptive material (188), including but not limited to being hydrophilic.

In versions where absorptive material (190) does not fully complement the curvature of window (175), a filler or spring-like material may be positioned between absorptive material (190) and window (175) to adequately provide form fit between absorptive material (190) and window (175). It will be appreciated that use of such a filler material may provide constant or substantially constant contact with window (175) as sheath (188) is advanced and retracted. In some versions, such a filler is in the form of a wiper blade or similar type of feature, an example of which is described in greater detail below.

In some versions, absorptive material (190) is sized to cover the entirety of window (175) when absorptive material (190) is advanced distally. In some other versions, absorptive material (190) is provided in two separate portions, each being smaller than window (175), such that the two separate portions move longitudinally or laterally along window (175) to clean window (175). In some such versions, the two portions of absorptive material (190) may move opposingly to meet in roughly the middle of window (175) or correspondingly across the surface of window (175) to wipe window (175). It should also be understood that absorptive material (190) may be retracted into a sheath, pocket, or other feature when absorptive material (190) is in the retracted position shown in FIG. 5A.

While absorptive material (190) is urged across window (175) by sheath (188) in the present example, other suitable features and mechanisms for advancing absorptive material (190) will be apparent to one of ordinary skill in the art in view of the teachings herein. By way of example only, rods extending along or integrated into shaft (130) may be used to push absorptive material (190) distally across window (175). In addition or in the alternative, absorptive material (190) may be pulled across the surface of window (175) through rods as discussed above or by any other suitable mechanisms as would be apparent to one of ordinary skill in the art in view of the teachings herein. In addition to pulling or pushing, absorptive material (190) may be operable to move laterally across window (175). In some instances, absorptive material (190) may be rolled across the surface of window (175) such that absorptive material (190) may rest atop of material to be absorbed. In some other versions, cleaning system (160) may be configured to provide varying normal forces with absorptive material (190) against window (175) as absorptive material (190) is translated, such that absorptive material (190) provides a dabbing action against window (175). As yet another merely illustrative variation, sheath (188) may be rotatable and non-translatable, with absorptive material (190) being thereby rotatable to be either positioned under window (175) or over window (175). Thus, when endoscope (110) is being used to visualize a site within the patient, absorptive material (190) may be under window (175) and out of view. When the operator wishes to clear window (175), the operator may rotate sheath (188) relative to shaft (130), and thereby wipe absorptive material (190) across window (175) to clear window (175). Other suitable variations of absorptive material (190) movement that may be used will be apparent to one of ordinary skill in the art in view of the teachings herein.

B. Exemplary Sheath with Bendable Wiper

In addition to or in the alternative to absorptive material (190), other structures may be used to clean window (175). FIG. 6 shows an exemplary sheath (278) with a wiper blade (280) extending inwardly from the distal end of sheath (278). Wiper blade (280) may be affixed to sheath (278) or, alternatively, may be integrally formed with sheath (278). In some versions, rather than using sheath (278), rods, wires, or other suitable components may be used to advance wiper blade (280). It should also be understood that wiper blade (280) may be pulled longitudinally across the surface of window (175); or urged laterally across window (175).

In some versions, wiper blade (280) may be movable between a proximal position and a distal position similar to the movement described above with respect to absorptive material (190) in reference to FIGS. 5A-5B. For instance, in the proximal position, wiper blade (280) may be proximal to window (175) and may move across window (175) as wiper blade (280) transitions to the distal position. Wiper blade (280) may be actuated using a feature such as button (180) described above, by grasping sheath (278) directly and translating sheath (278), or in any other suitable fashion.

In some versions, multiple wiper blades (280) are used or wiper blade (280) is formed of two separate portions of either equal or differing sizes. The two portions may be moved longitudinally or laterally along window (175) to clean window (175). By way of example only, wiper blade (280) may comprise two or more blades that run parallel to each other transversely relative to shaft (130), similar to a conventional automotive windshield wiper. As another merely illustrative example, the two portions of wiper blade (280) may move in opposing directions to meet in roughly the middle of window (175) or may move correspondingly across the surface of window (175) to wipe window (175). Other suitable variations of wiper blade (280) movement will be apparent to those of ordinary skill in the art in view of the teachings herein.

Wiper blade (280) of the present example is hydrophobic and resilient. By way of example only, wiper blade (280) may be made of silicone, rubber, or some other non-porous material. Wiper blade (280) may also be configured to conform to the shape of window (175). As shown in FIG. 6, wiper blade (280) is configured to resiliently bear against window (175) as wiper blade (280) is advanced and retracted over window (175). As wiper blade (280) is advanced distally across window (175), the free end of wiper blade (280) bends toward the proximal end of endoscope (110). As wiper blade (280) is retracted proximally across window (175), the free end of wiper blade (280) bends toward the distal end of endoscope (110). It should be understood that wiper blade (280) may have various configurations. By way of example only, wiper blade (280) may extend along a substantially straight line, from one lateral side of window (175) to the other lateral side of window (175). In some other versions, a wiper blade (380) has a projecting leading edge (382), as shown in FIG. 7. Such a configuration may assist with pushing fluid and debris outwardly relative to window (175), rather than just pushing fluid and debris distally relative to window (175). As a similar variation, wiper blade (380) may be generally triangular, with a point of wiper blade (380) projecting distally. Other suitable configurations for wiper blade (380) will be apparent to those of ordinary skill in the art in view of the teachings herein.

FIG. 8A shows an alternative version of wiper (476) with sheath (478). Wiper (476) of this example comprises a shape memory material extending further longitudinally along sheath (478) than wiper blade (280) of FIG. 6. The underside of wiper (476) may include a wiper blade and/or an absorptive material, etc. In the illustrated version, sheath (478) and wiper (476) tougher define an aperture (479) that is configured to receive the distal end of an endoscope (410) as shown in FIG. 8B. Wiper (476) may comprise nitinol, silicone, and/or any of the other materials referred to herein (among others). As seen in FIG. 8A, wiper (476) resiliently biased to extend downwardly, such that wiper (476) drags against the window of endoscope (410) as wiper (476) is translated longitudinally relative to endoscope (410). By way of example only, sheath (478) and wiper (476) may be held at the proximal position shown in FIG. 8B during normal operation of endoscope (410); and then be advanced distally to the position shown in FIG. 8A to wipe blood, mucus, debris, etc. from endoscope (410). Sheath (478) and wiper (476) may be advanced distally in any of the ways described herein or in any other suitable fashion.

It should be understood that any of the wipers (280, 380, 476) described herein may be combined with absorptive material (190) described herein. By way of example only, wiper (280, 380, 476) may be positioned distal to absorptive material (190), such that absorptive material (190) absorbs fluid left behind after wiper (280, 380, 476) passes over window (175). Other suitable combinations will be apparent to those of ordinary skill in the art in view of the teachings herein.

C. Exemplary Sheath with Combination of Wiping, Washing, and/or Suction

In some instances, mechanical wiping of a window may not be sufficient to remove all material or debris from a window (175) of an endoscope (110). Thus, it may be desirable to provide additional cleaning features for the window (175). FIGS. 9-10 show an exemplary wash and suction device (550) operable to dispense a fluid wash, provide suction, and actuate a wiper (576) to clean window (175) during an endoscopic medical procedure. Wash and suction device (550) comprises a sheath (530) that defines a lumen (532) sized to receive a combination of endoscope (110) and sheath (188) of cleaning system (160). In this example, the distal end of sheath (188) includes a wiper (576), which may be configured similar to any of the wipers (280, 380, 476) described herein or in any other suitable fashion.

Sheath (530) of the present example further comprises a fluid conduit (580) and a suction conduit (582). Fluid conduit (580) has an arcuate profile and is positioned over window (175). Suction conduit (582) has a generally larger arcuate profile and is positioned under window (175). Of course, suction conduit (582) may be smaller than fluid conduit (580) or they may be equal in size. Furthermore, one of conduits (580, 582) may be omitted in some other versions. Fluid conduit (580) is in communication with a fluid source (584) and is operable to dispense a fluid (e.g., saline, pressurized air, etc.) across window (575) and/or wiper (576). Suction conduit (582) is in communication with a suction source (586) and is operable to vacuum fluid or debris through suction conduit (586). Fluid source (584) and suction source (586) may be powered using a variety of mechanisms. For instance, fluid source (584) may include a pressurized bag or basin for driving fluid, a pump, a gravity bag, or hand pumps. Fluid source (584) may be controllable through directly manipulating the above mentioned mechanisms or through the use of foot pedals in communication with fluid source (584). Suction source (586) may include a manual or motor driven pump, etc. Suction source (586) may similarly be controllable through directly manipulating the above mentioned mechanisms or through the use of foot pedals and/or other components in communication with suction source (586). In some versions, at least part of wash and suction device (550) is constructed in accordance with at least some of the teachings of U.S. patent application Ser. No. [ATTORNEY DOCKET NO. ACC5053USPSP.0600265], entitled “Apparatus for Flushing Angled Window of Endoscope,” filed on even date herewith, the disclosure of which is incorporated by reference herein.

In some versions, a combination of a fluid wash and mechanical wipe may be provided by wash and suction device (550) and wiper (576). For instance, fluid conduct (580) may dispense liquid across window (175) and wiper (576) may subsequently or simultaneously wipe window (175) further cleaning window (175). In addition to or in the alternative, absorptive material (190) such as one shown in FIG. 3 may also wipe window (175), thereby providing an absorptive material operable to absorb at least a portion of the fluid wash provided by wash and suction device (550).

Similarly, suction through suction conduit (586) may be used in conjunction with a mechanical wipe. In particular, as wiper (576) and/or absorptive material (190) wipes debris from window (175), suction conduit (582) may provide sufficient suction to vacuum the debris or material. In addition or in the alternative, suction conduit (586) may suck material or debris directly from absorptive material (190), which may have absorbed fluid or material. Suction conduit (586) may thus be used to remove debris from absorptive material (190) that might otherwise hinder the absorption provided by absorptive material (190). Suction may thus be used to clean window (175), to clean absorptive material (190), and/or to clean the endoscopic field. It will be appreciated that both suction and a fluid wash may be used together with either or both of wiper (576) or absorptive material (190).

In any of the examples described herein, a hydrophobic coating may be applied to window (175), thereby making it easier to remove material from window (175) with the numerous features described herein. Other suitable variations will be apparent to those of ordinary skill in the art in view of the teachings herein.

IV. Miscellaneous

It should be understood that any of the examples described herein may include various other features in addition to or in lieu of those described above. By way of example only, any of the examples described herein may also include one or more of the various features disclosed in any of the various references that are incorporated by reference herein.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices described above may have application in conventional medical treatments and procedures conducted by a medical professional, as well as application in robotic-assisted medical treatments and procedures. By way of example only, various teachings herein may be readily incorporated into a robotic surgical system such as the DAVINCI™ system by Intuitive Surgical, Inc., of Sunnyvale, Calif. Similarly, those of ordinary skill in the art will recognize that various teachings herein may be readily combined with various teachings of U.S. Pat. No. 6,783,524, entitled “Robotic Surgical Tool with Ultrasound Cauterizing and Cutting Instrument,” published Aug. 31, 2004, the disclosure of which is incorporated by reference herein.

Versions described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by a user immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/we claim:

1. An apparatus comprising:
(a) a sheath configured to receive an endoscope shaft, wherein the sheath is operable to translate relative to the endoscope shaft; and
(b) a wiper in communication with the distal end of the sheath, wherein the wiper is configured to move across at least a portion of a window of the endoscope in response to movement of the sheath relative to the endoscope shaft.

2. The apparatus of claim 1, wherein the wiper comprises a distally projecting taper.

3. The apparatus of claim 1, wherein the wiper is shaped to complement a contour of the window.

4. The apparatus of claim 1, wherein the wiper comprises a shape memory material.

5. The apparatus of claim 1, further comprising a filler material between the window and the wiper.

6. The apparatus of claim 1, wherein the sheath is configured to translate longitudinally relative to the endoscope shaft.

7. The apparatus of claim 1, wherein the wiper is configured to translate longitudinally relative to the window.

8. The apparatus of claim 1, wherein the wiper comprise an absorptive material.

9. The apparatus of claim 1, wherein the wiper comprises a hydrophobic material.

10. The apparatus of claim 1, further comprising a wash assembly operable to deliver a wash fluid to the window.

11. The apparatus of claim 1, further comprising a suction assembly operable to vacuum fluid from the window.

12. The apparatus of claim 1, further comprising a pressurized gas source operable to apply a pressurized gas across the window.

13. The apparatus of claim 1, wherein the sheath comprises an absorptive material.

14. The apparatus of claim 1, further comprising a resilient feature operable to urge the wiper toward the window.

15. The apparatus of claim 1, wherein the wiper comprises a multi-finned wiper blade.

16. An apparatus comprising:
(a) a sheath configured to receive an endoscope shaft, wherein the sheath is operable to translate relative to the endoscope shaft, wherein the sheath defines a longitudinal axis; and
(b) a hydrophobic wiper blade in communication with the distal end of the sheath, wherein the wiper blade is configured to move across at least a portion of a window of the endoscope in response to movement of the sheath relative to the endoscope shaft, wherein the hydrophobic wiper blade is configured to move along a path that is oblique relative to the longitudinal axis of the sheath as the wiper blade passes along the window.

17. The apparatus of claim 16, where the wiper further comprises a leading tip.

18. The apparatus of claim 16, further comprising a camming button feature operable to drive the sheath distally.

19. The apparatus of claim 16, wherein the wiper comprises a shape memory material.

20. A method of cleaning an endoscope window using a cleaning system comprising a sheath and a wiper blade, the method comprising:
(a) inserting the endoscope into the sheath of the cleaning system; and
(b) advancing the wiper blade along at least a portion of the window, thereby cleaning the window.
